(19) United States
(12) Patent Application Publication
(10) Pub. No.: US 2023/0102555 A1
(43) Pub. Date: Mar. 30, 2023

(54) MOBILE QT ANALYSIS INTEGRATION

(71) Applicant: AliveCor, Inc., Mountain View, CA (US)

(72) Inventors: Arush Pandya, Cranford, NJ (US); Aman Bhatti, Chester, NJ (US)

(21) Appl. No.: 17/955,336

(22) Filed: Sep. 28, 2022

Related U.S. Application Data
(60) Provisional application No. 63/249,524, filed on Sep. 28, 2021.

(51) Int. Cl. G16H 10/60; A61B 5/00; A61B 5/36; G16H 50/20

(52) U.S. Cl. CPC G16H 10/60; A61B 5/0006; A61B 5/36; A61B 5/7435; A61B 5/743; G16H 50/20

(57) ABSTRACT

Embodiments of the present disclosure relate to providing GUIs for visualizing ECG data and integrated QT analysis results. A system may comprise an electrocardiogram (ECG) monitoring device to measure an ECG of a user to generate ECG data and transmit the ECG data. The system may further comprise a cloud analysis platform to receive the ECG data, perform a QT analysis based on the ECG data to generate QT analysis results, and transmit the QT analysis results. The system may further comprise a computing device to receive the QT analysis results and provide a graphical user interface (GUI) to visualize the ECG data with the QT analysis results integrated therein.

### CROSS-REFERENCE TO RELATED APPLICATIONS

This application claims the benefit of U.S. Provisional Application No. 63/249,524, filed Sep. 28, 2021, and entitled MOBILE QT ANALYSIS INTEGRATION, the contents of which are hereby incorporated by reference in their entirety.

### BACKGROUND

It is estimated that by 2030, over 23 million people will die from cardiovascular diseases annually. Cardiovascular diseases are prevalent in across populations of first world as well as third world countries and regardless of socioeconomic status. Monitoring of cardiovascular function can aid in the treatment and prevention of cardiovascular disease. For example, a patient with A-fib (or other type of arrhythmia) can be monitored for extended periods of time to manage the disease using a Holter monitor or other ambulatory electrocardiography device. Such devices can continuously monitor the electrical activity of the cardiovascular system for e.g., at least 24 hours. Such monitoring can be critical in detecting conditions such as acute coronary syndrome (ACS), among others.

The mammalian heart generates and conducts an electric current that signals and initiates the coordinated contraction of the heart. In humans, an electrical signal is produced by a portion of the heart known as the SA node. After being generated by the SA node, the electric current travels throughout the myocardium in a manner that is predictable in a healthy heart.

In general, an electrocardiogram (ECG) is a graphic representation of the electric conduction of the heart over time as projected on the surface of the body. An ECG is typically displayed on a graph having an x and y axis. Typically, the x-axis of an ECG displays time and the Y-axis of an ECG displays the electric potential (in millivolts) of an electric current that is conducted through the heart during normal cardiac function.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a pictorial representation of a prior art electrocardiograph having 10 electrodes positioned on a patient's body for taking a prior art 12-lead electrocardiogram, in accordance with some embodiments of the present disclosure.

FIG. 2 is a pictorial representation of a chest showing an example of electrode placement on the chest for taking a prior art 12-lead electrocardiogram, in accordance with some embodiments of the present disclosure.

FIG. 3 illustrates an example Lead I annotated to show PQRST waves generated by a 12-lead electrocardiograph, in accordance with some embodiments of the present disclosure.

FIG. 4 shows an example 12-lead electrocardiogram in a conventional format, in accordance with some embodiments of the present disclosure.

FIG. 5 illustrates an exemplary system, in accordance with some embodiments of the present disclosure.

FIGS. 6A-6C illustrate exemplary graphical user interfaces (GUIs) for visualizing ECG data and integrated QT analysis results, in accordance with some embodiments of the present disclosure.

FIGS. 7A-7F illustrate exemplary GUIs for visualizing ECG data and integrated QT analysis results, in accordance with some embodiments of the present disclosure.

FIGS. 8A and 8B illustrate exemplary GUIs for visualizing ECG data and integrated QT analysis results, in accordance with some embodiments of the present disclosure.

FIGS. 9A-9D illustrate exemplary GUIs for accessing ECG data and integrated QT analysis results directly from a cloud analysis site, in accordance with some embodiments of the present disclosure.

FIGS. 10A-11 illustrate exemplary GUIs for accessing ECG data and integrated QT analysis results directly from a cloud analysis site, and for requesting/performing an overread on ECG data and integrated QT analysis results, in accordance with some embodiments of the present disclosure.

FIGS. 12A-12E illustrate an ECG monitoring device, in accordance with some embodiments of the present disclosure.

FIG. 13 illustrates a method for providing GUIs for visualizing ECG data and integrated QT analysis results, in accordance with some embodiments of the present disclosure

FIG. 14 is a block diagram of an example computing device that may perform one or more of the operations described herein, in accordance with some embodiments of the present disclosure.

### DETAILED DESCRIPTION

It is to be understood that the present disclosure is not limited in its application to the details of construction, experiments, exemplary data, and/or the arrangement of the components set forth in the following description. The embodiments of the present disclosure are capable of other embodiments or of being practiced or carried out in various ways. Also, it is to be understood that the terminology employed herein is for purpose of description and should not be regarded as limiting.

An electrocardiogram (ECG) provides a number of ECG waveforms that represent the electrical activity of a person's heart. An ECG monitoring device may comprise a set of electrodes for recording these ECG waveforms (also referred to herein as “taking an ECG”) of the patient's heart. The set of electrodes may be placed on the skin of the patient in multiple locations and the electrical signal (ECG waveform) recorded between each electrode pair in the set of electrodes may be referred to as a lead. Varying numbers of leads can be used to take an ECG, and different numbers and combinations of electrodes can be used to form the various leads. Example numbers of leads used for taking ECGs are 3, 5, and 12 leads.

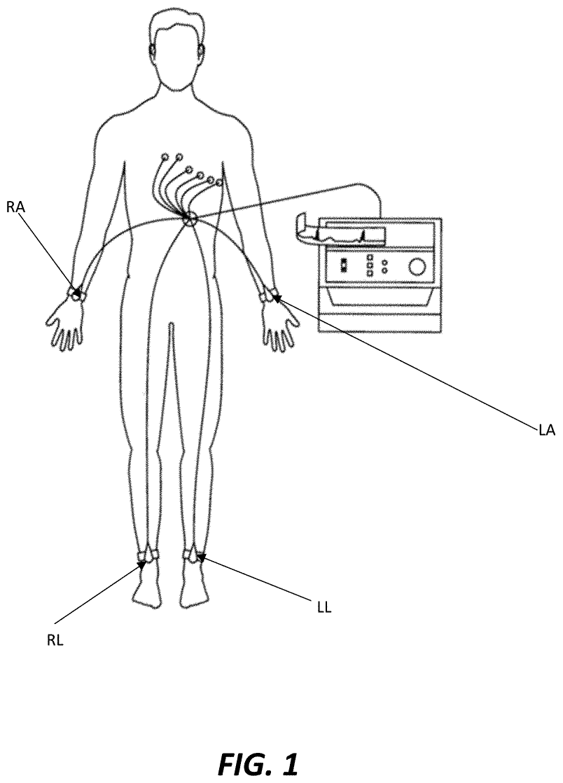

FIG. 1 is a pictorial representation of the 10 electrodes of a conventional ECG monitoring device being placed on the patient for obtaining a standard 12-lead ECG. The electrode placed on the right arm is commonly referred to as RA. The electrode placed on the left arm is referred to as LA. The RA and LA electrodes are placed at the same location on the left and right arms, preferably but not necessarily near the wrist. The leg electrodes can be referred to as RL for the right leg and LL for the left leg. The RL and LL electrodes are placed on the same location for the left and right legs, preferably but not necessarily near the ankle.

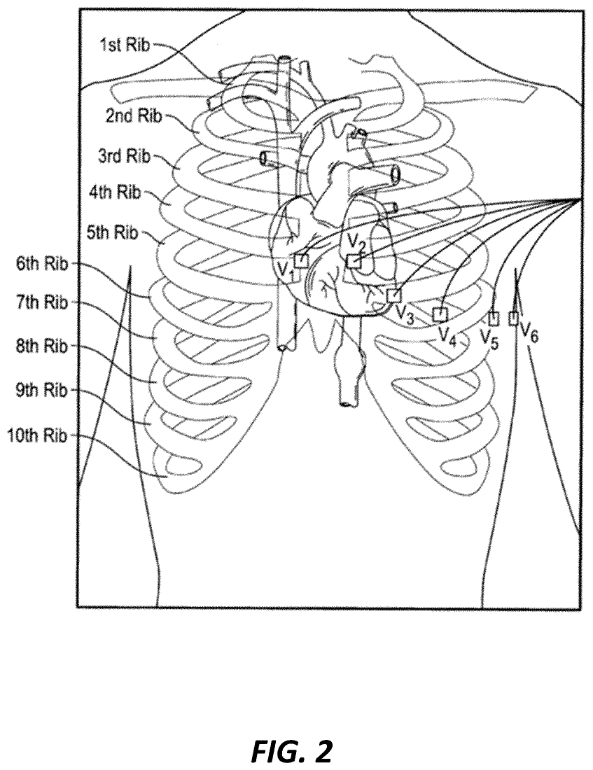

FIG. 2 illustrates the placement of the six electrodes on the chest with the six electrodes being labeled V₁, V₂, V₃, V₄, V₅, and V₆ respectively. V₁ is placed in the fourth intercostal space, for example between ribs 4 and 5, just to the right of the sternum. V₂ is placed in the fourth intercostal space, for example between ribs 4 and 5, just to the left of the sternum. V₃ is placed in the fifth intercostal space midway between electrodes V₂ and V₄. V₄ is placed in the fifth intercostal space between ribs 5 and 6 on the left mid-clavicular line. V₅ is placed horizontally even with V₄ on the left anterior axillary line. V₆ is placed horizontally even with V₄ and V₅ on the left mid-axillary line.

The electrocardiograph then calculates and outputs three limb lead waveforms. Limb leads I, II, and III are bipolar leads having one positive and one negative pole. Lead I is the voltage between the left arm (LA) and right arm (RA), e.g. I=LA−RA. Lead II is the voltage between the left leg (LL) and right arm (RA), e.g., II=LL−RA. Lead III is the voltage between the left leg (LL) and left arm (LA), e.g. III=LL−LA. Leads I, II and III are commonly referred to as “limb leads.”

Unipolar leads also have two poles; however, the negative pole is a composite pole made up of signals from multiple other electrodes. In a conventional cardiograph for obtaining a 12-lead ECG, all leads except the limb leads are unipolar (aVR, aVL, aVF, V₁, V₂, V₃, V₄, V₅, and V₆). Augmented limb leads (aVR, aVL, and aVF) view the heart from different angles (or vectors) and are determined from electric potential differences between one of RA, LA, and LL, and a composite comprising of two of RA, LA, and LL. Thus, three electrodes positioned at RA, LA, and LL will sense aVR, aVL, and aVF simultaneously based on the above relationships. Which is to say that while leads, I, II, and III each require input from only two electrodes, and aVR, aVL, and aVF may require input from three electrodes positioned at RA, LA, and LL.

For example, the augmented vector right (aVR) positions the positive electrode on the right arm, while the negative electrode is a combination of the left arm electrode and the left leg electrode, which “augments” the signal strength of the positive electrode on the right arm. Thus the augmented vector right (aVR) is equal to RA−(LA+LL)/2 or −(I+II)/2. The augmented vector left (aVL) is equal to LA−(RA+LL)/2 or (I−II)/2. The augmented vector foot (aVF) is equal to LL−(RA+LA)/2 or (II−I)/2.

In one embodiment, the six electrodes on the chest of the patient are close enough to the heart that they do not require augmentation. A composite pole called Wilson's central terminal (often symbolized as CT_{W}, V_{W}, or WCT) is used as the negative terminal. Wilson's central terminal is produced by connecting the electrodes RA, LA, and LL together, via a simple resistive network, to give an average potential across the body, which approximates the potential at an infinite distance (i.e. zero). Wilson's central terminal, WCT, is calculated as (RA+LA+LL)/3.

The ECG waveforms (each one corresponding to a lead of the ECG) recorded by the ECG monitoring device may comprise data corresponding to the electrical activity of the person's heart. A typical heartbeat may include several variations of electrical potential, which may be classified into waves and complexes, including a P wave, a QRS complex, a T wave, and a U wave among others, as is known in the art. Stated differently, each ECG waveform may include a P wave, a QRS complex, a T wave, and a U wave among others, as is known in the art. The shape and duration of these waves may be related to various characteristics of the person's heart such as the size of the person's atrium (e.g., indicating atrial enlargement) and can be a first source of heartbeat characteristics unique to a person. Each wave or a complex of multiple waves (i.e. the QRS complex) is associated with a different phase of the heart's depolarization and repolarization. The ECG waveforms may be analyzed (typically after standard filtering and “cleaning” of the signals) for various indicators that are useful in detecting cardiac events or status, such as cardiac arrhythmia detection and characterization. Such indicators may include ECG waveform amplitude and morphology (e.g., QRS complex amplitude and morphology), R wave-ST segment and T wave amplitude analysis, and heart rate variability (HRV), for example.

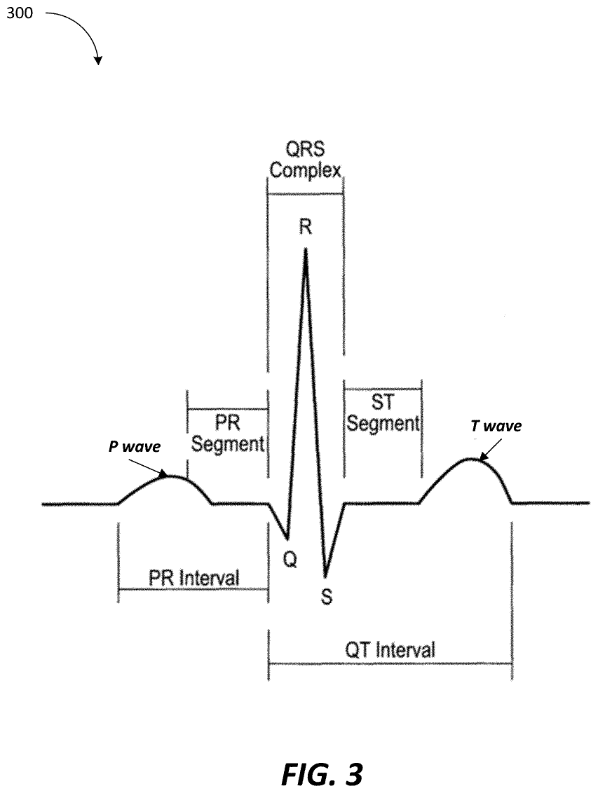

FIG. 3 illustrates an example Lead I annotated to show P, QRS, and T waves/complexes generated by a 12-lead electrocardiograph. Typically, an ECG of a normal beating heart has a predictable wave-form in each of the twelve ECG leads. ECG portions between two waves are referred to as segments and ECG portions between more than two waves are referred to as intervals. For example, the ECG portion between the end of the S wave (part of QRS complex) and the beginning of the T wave is referred to as the ST segment while the portion of the ECG between the beginning of the Q wave (part of QRS complex) and the end of the T wave is referred to as the QT interval.

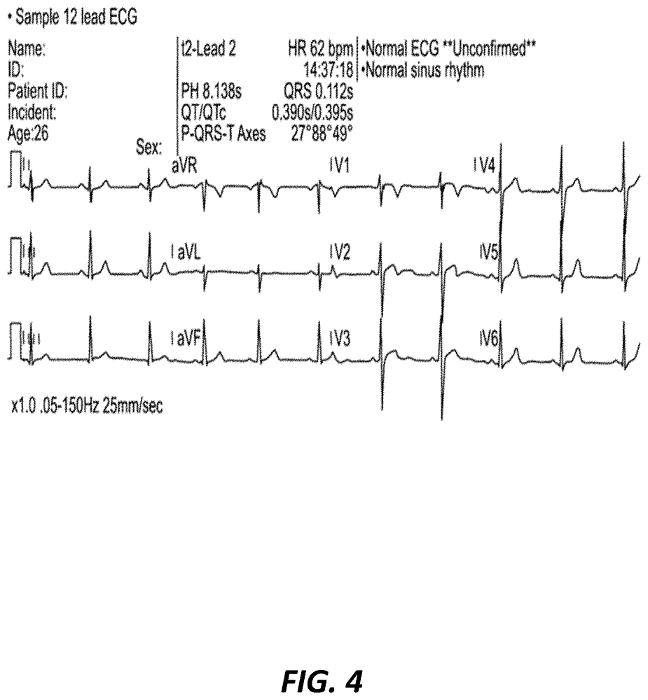

FIG. 4 shows an example 12-lead electrocardiogram in a conventional format. As shown in FIG. 4, for standard ECG waveform tracing, twelve ECG leads are displayed individually on an X and Y axis, wherein the Y-axis represents time and the X-axis represents voltage. In these tracings, all twelve ECG waveforms are aligned with respect to their X-axes. That is, the P, QRS, and T waveforms of all the leads all occur at the same time along the X-axis of each of the respective tracings. For example, in a traditional ECG waveform tracing, if a QRS complex occurs at 1 second on the X-axis in the lead I waveform tracing, a QRS complex occurs at 1 second in each of the other eleven ECG waveforms (i.e. leads II, III, aVR, aVL, aVF, V₁, V₂, V₃, V₄, V₅, and V₆).

The standard time aligned format allows health care providers to more easily obtain information from the twelve sensed ECG waveforms. In the traditional ECG tracing, time alignment is facilitated by virtue of the waveforms being sensed simultaneously by the ten electrodes of the traditional ECG that are all simultaneously positioned on the skin of the individual whose ECG is sensed. That is, because all twelve ECG leads of a traditional ECG are sensed simultaneously, time-alignment is achieved by simply displaying all of the waveforms together on identical axes.

It should be noted that a set of two or more leads may be analyzed to derive information to generate a full, 12-lead ECG. Such transformation may be performed using a machine learning model (e.g., a neural network, deep-learning techniques, etc.). The machine learning model may be trained using 12-lead ECG data corresponding to a population of individuals. The data, before being input into the machine learning model, may be pre-processed to filter the data in a manner suitable for the application. For example, data may be categorized according to height, gender, weight, nationality, etc. before being used to train one or more machine learning models, such that the resulting one or models are finely-tuned the specific types of individuals. In a further embodiment, the machine learning model may be further trained based on a user's own ECG data, to fine-tune and personalize the model even further to decrease any residual synthesis error.

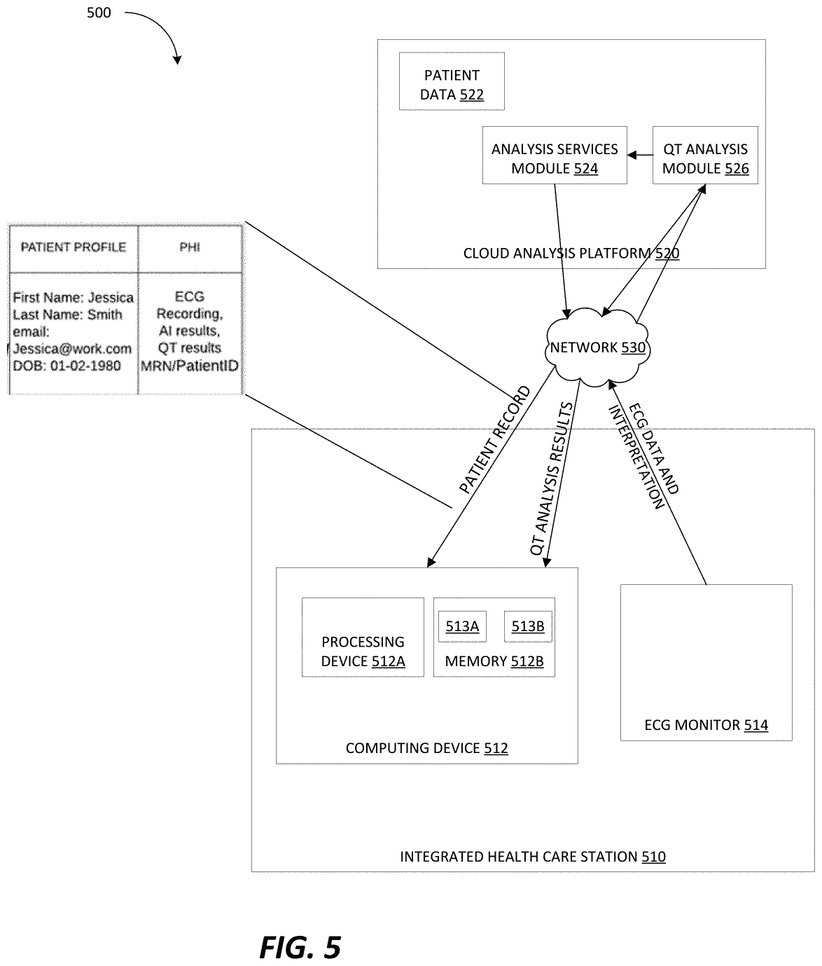

FIG. 5 illustrates a system 500 in accordance with some embodiments of the present disclosure. The system 500 may comprise an integrated health care station 510 for managing/visualizing ECG and QT analysis results and a cloud computing/storage service 520. The integrated health care station 510 may be hardware provided by a healthcare provider facility and may comprise a computing device 512 and an ECG monitoring device 514. The computing device 512 may include hardware such as processing device 512A (e.g., processors, central processing units (CPUs)), memory 512B (e.g., random access memory (RAM), storage devices (e.g., hard-disk drive (HDD), solid-state drive (SSD), etc.), and other hardware devices (e.g., sound card, video card, etc.). In some embodiments, memory 512B may be a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices. Memory 512B may be configured for long-term storage of data and may retain data between power on/off cycles of the computing device 512. The computing device 512 may comprise any suitable type of computing device or machine that has a programmable processor including, for example, server computers, desktop computers, laptop computers, tablet computers, smartphones, set-top boxes, etc. In some examples, the computing device 512 may comprise a single machine or may include multiple interconnected machines (e.g., multiple servers configured in a cluster). Although illustrated as being integrated together on board the integrated health care station 510, this is not a limitation and the integrated health care station 510 may be implemented with the ECG monitoring device 514 and the computing device 512 as separate devices that communicate with each other via a wired or wireless connection as discussed herein.

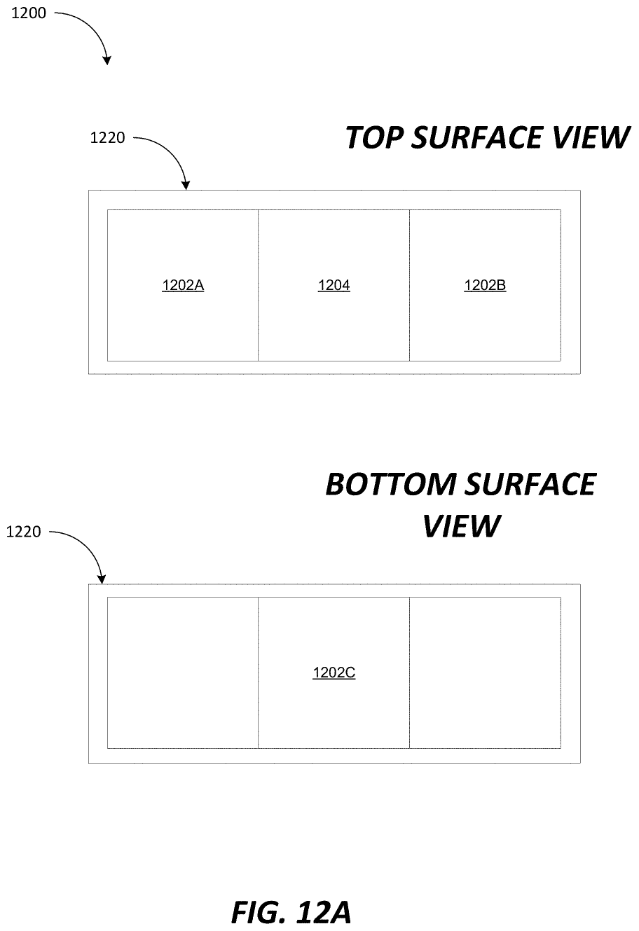

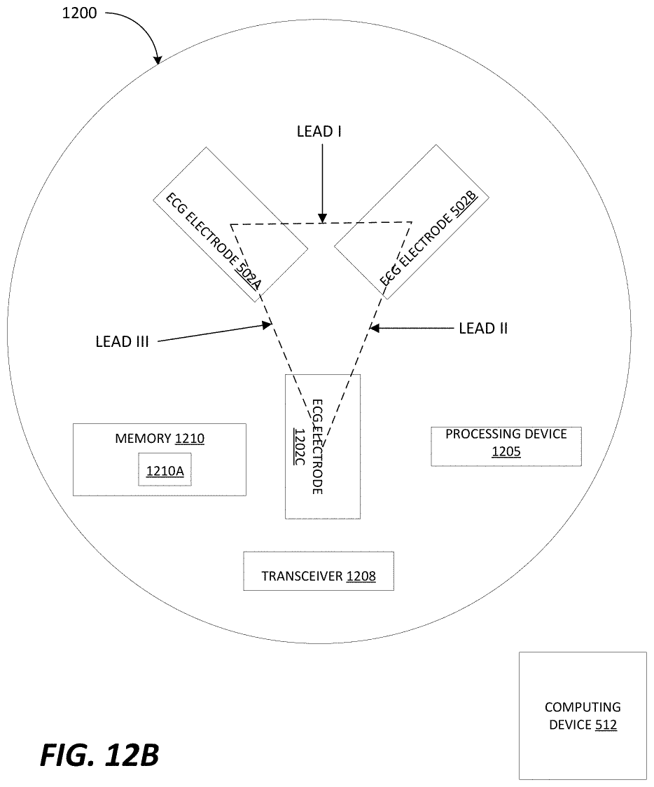

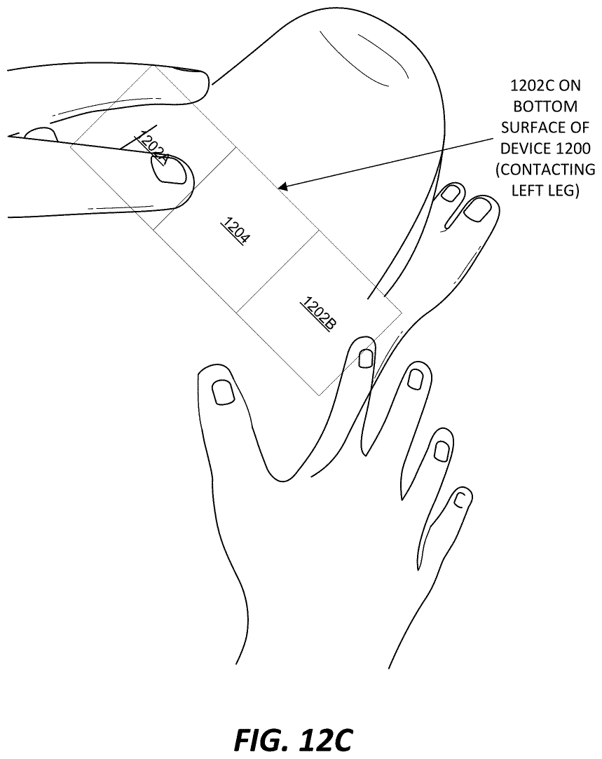

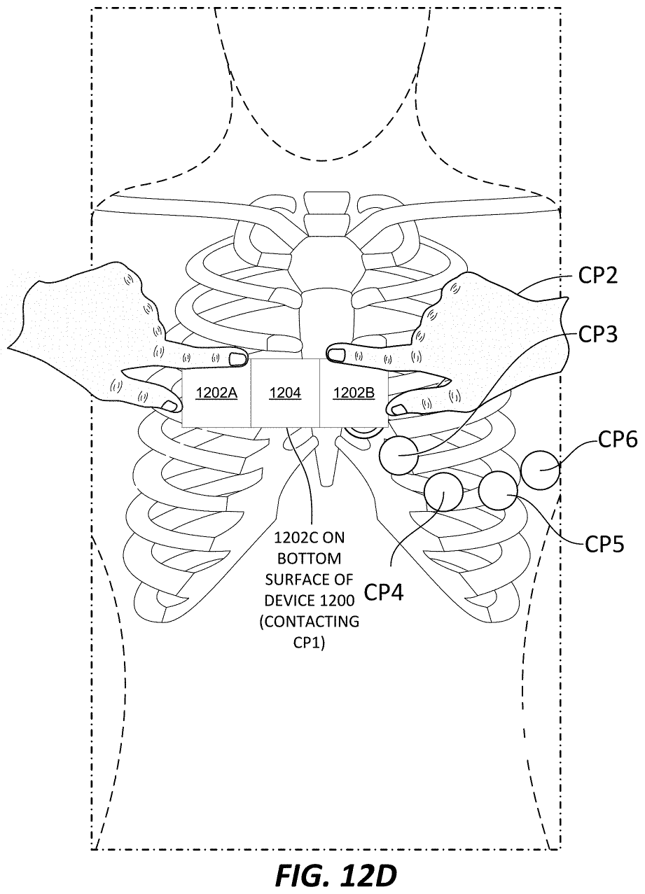

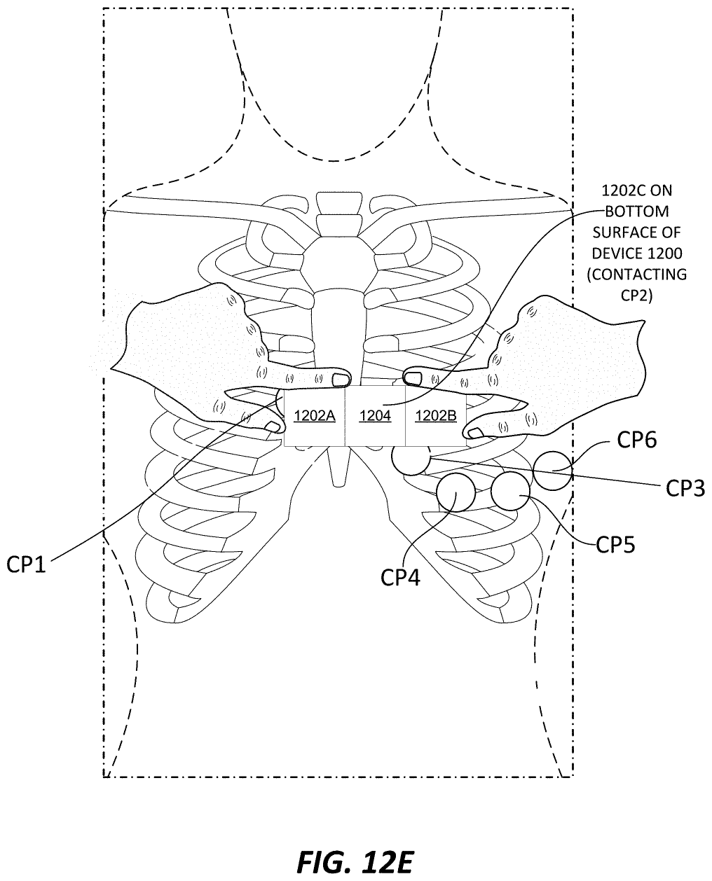

The ECG monitoring device 514 may comprise any appropriate ECG measuring hardware/software to take an ECG of a patient such as the conventional 10 electrode ECG monitoring device illustrated in FIG. 1, or a reduced form factor/reduced lead set device such as the Kardia Pro™ as discussed in further detail with respect to FIGS. 12A-12C. The ECG monitoring device 514 may further include software/logic corresponding to a machine learning (ML) module for synthesizing one or more leads of the ECG e.g., based on one or more leads measured by the electrodes of the ECG monitoring device 514 as discussed herein. The ECG monitoring device 514 may also include software/logic corresponding an artificial intelligence (AI) interpretation module for determining interpretations based on the measured ECG data.

The cloud analysis platform 520 may be any appropriate cloud service such as Amazon S3™, for example and may store patient data 522 for a large number of patients including date of birth, height, weight, sex, and other health conditions etc. The patient data 522 for each patient may also include current and previous results including measured ECG, AI interpretations, and results of QT analysis performed by the QT analysis module 526 as discussed in further detail herein. The cloud analysis platform 520 may also host/execute applications such as the analysis services module 524 and the QT analysis module 526.

The integrated health care station 510 and the cloud analysis platform 520 may be coupled to each other (e.g., may be operatively coupled, communicatively coupled, may communicate data/messages with each other) via network 530. Network 530 may be a public network (e.g., the internet), a private network (e.g., a local area network (LAN) or wide area network (WAN)), or a combination thereof. In one embodiment, network 530 may include a wired or a wireless infrastructure, which may be provided by one or more wireless communications systems, such as a Wi-Fi hotspot connected with the network 530 and/or a wireless carrier system that can be implemented using various data processing equipment, communication towers (e.g., cell towers), etc. The network 530 may carry communications (e.g., data, message, packets, frames, etc.) between the integrated health care station 510 and the cloud computing/storage service 520.

A healthcare professional may log into the integrated health care station 510 and provide identifying information about a patient such as e.g., their patient MRN/ID. The healthcare professional may then take an ECG of the patient using the ECG monitoring device 514 to generate ECG data of the patient. Upon generating ECG data of the patient, the ECG monitoring device 514 may also generate an interpretation based on the ECG data. The ECG monitoring device 514 may utilize an AI interpretation module to generate the interpretation. The ECG monitoring device 514 may transmit the ECG data and interpretation (along with the patient's identifying information) to the cloud analysis platform 520. In some embodiments, the ECG monitoring device 514 may transmit the ECG data and interpretation (along with the patient's identifying information) to the computing device 512 for transmission to the cloud analysis platform 520.

Upon receiving the ECG data and interpretation (along with the patient's identifying information), the cloud analysis platform 520 may execute the QT analysis module 526 in order to perform a QT analysis and generate QT analysis results. The QT analysis results may include the patient's QT and QTcF values. The analysis services module 524 may transmit the QT analysis results to the computing device 512. The memory 512B of the computing device 512 may include an ECG and QT analysis/management module 513A which may include logic for providing an improved graphical user interface (GUI) for visualizing the ECG data with the QT analysis results integrated as discussed in further detail herein. The GUI for visualizing the ECG data with integrated QT analysis results may be displayed on a display of the computing device 512.

In some embodiments, the QT analysis module 526 may comprise a machine learning (ML) model that predicts the QT interval. In one example, the QT analysis module 526 may comprise a deep neural network (DNN) for predicting the QT interval, however any appropriate ML model may be used. The QT analysis module 526 may process the recorded ECG data with an average beat algorithm and the input to the DNN may be the average beat in the form of a 2×450 length signal in millivolts. The average beat signal may first be clamped via a hyperbolic tangent function before going through an initial convolutional layer (e.g., 2D convolution, leaky rectified linear unit, max pooling) before being processed by three separate passthrough stages (two separate 2D convolutional, leaky rectified linear unit, max pooling, etc.), with the passthrough input added (via a trainable parameter) and dropout applied before downsampling, pooling, and convolution. After the final passthrough, the signal is sent through two separate feed-forward network layers outputting a probability vector for the output class of a given QT value, whose expectation value is taken to as the predicted QT interval. The loss function for training is a sum of a cross entropy term of the probability vector and the target QT interval, and a root mean error squared QT interval average. It should be noted that the ML model may utilize (and be trained on) any appropriate number and combination of leads. In some embodiments, the ML model may be trained to predict the QT interval based on a combination of leads I, II, or III and any appropriate v lead. For example, the ML model may be trained based on lead I and v5, lead II and v5, or lead III and v5 (or any other appropriate v lead).

The DNN may be trained using annotated standard 12-lead ECG data of a variety of patients taken in the same position in which the user was in when the ECG is taken. In some embodiments, the ECG signals may be preprocessed before training. For example, a bandpass filter may be applied to simulate the ambulatory bandwidth (0.1-40 Hz) of the ECG monitoring device 500 and any appropriate filtering algorithm may be applied before processing the resulting 12-lead ECGs with an average beat algorithm for leads I and II. The source ECGs comprising the training data may be bucketed based on QT interval and randomly selecting ECGs within randomly selected buckets. Because the ECG signal received from the user may include leads that were generated using e.g., a separate lead conversion ML algorithm, in some embodiments, the DNN may be further adapted using additional training data including synthesized leads.

The analysis services module 524 may use the patient identifier (e.g., MRN/ID provided along with the ECG data by the ECG monitoring device 514) to add the QT analysis results, the ECG data, and the interpretation to a corresponding entry in the patient data 522. The analysis services module 524 may also append a limited set of patient information (e.g., first and last name, email address, and date of birth in the example of FIG. 5) to the QT analysis results, the ECG data, and the interpretation to generate a patient record. The cloud analysis platform 520 may provide a portal via which a health care provider can access the patient record and other patient data 522 of the patient via the computing device 512 of the integrated health care station 510 as discussed in further detail herein.

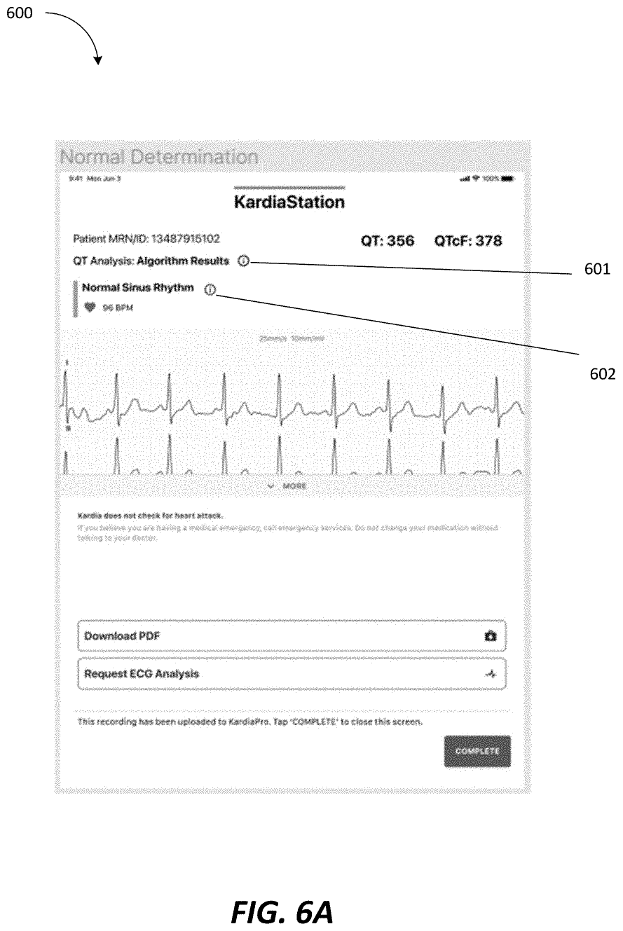

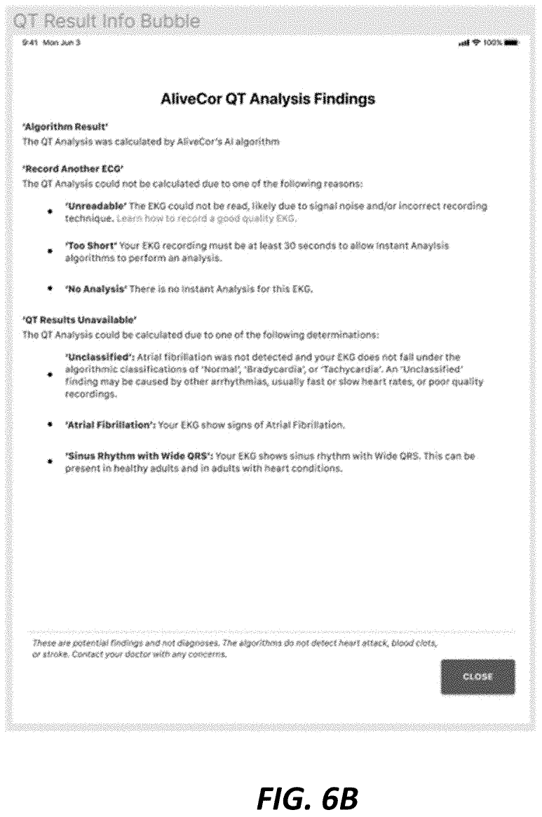

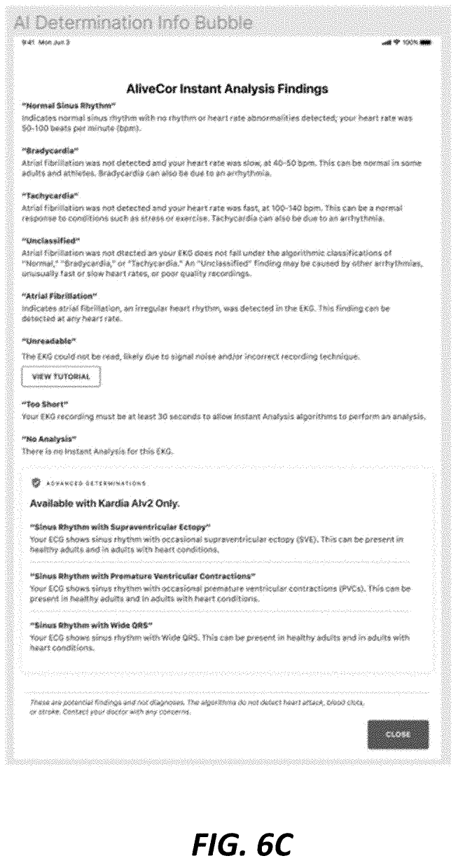

FIG. 6A illustrates an example GUI 600 for visualizing an ECG measurement with integrated QT analysis results in accordance with some embodiments of the present disclosure. As shown in FIG. 6A, the GUI 600 may provide, in addition to the patient's ECG, a patient identifier such as an MRN/ID of the patient, an indication of the patient's QT and QTcF values, an indication of an origin of the QT analysis results (e.g., FIG. 6A indicating that the results are algorithm-based), and an indication of the results of the QT analysis performed by the cloud analysis platform 520 (e.g., FIG. 6A indicating a normal sinus rhythm). The GUI indications of the origin of the QT analysis results and the QT analysis results themselves may indicate any of a number of statuses as discussed below with respect to FIGS. 6B and 6C. The GUI 600 may also provide options to download the results of the QT analysis as a PDF and request analysis of the patient's ECG e.g., by a physician or other health care professional (referred to as an overread).

The GUI indications of the origin of the QT analysis results and the QT analysis results may include icons (shown as an “i” within a circle in FIG. 6A) 601 and 602 respectively which may be interacted with by a user to obtain more information about those indications respectively. When a user interacts with the icon 601 tied to the indication of the origin of the QT analysis results (e.g., by clicking on it), they may be provided information on the various possible different statuses of the indication of the origin of the QT analysis results. The information may be provided in a pop-up screen as depicted in FIG. 6B, which illustrates the various different statuses. When a user interacts with the icon 602 tied to the indication of the QT analysis results, they may be provided information on the various possible different statuses of the indication of the QT analysis results. The information may be provided in a pop-up screen as depicted in FIG. 6C, which illustrates the various different statuses.

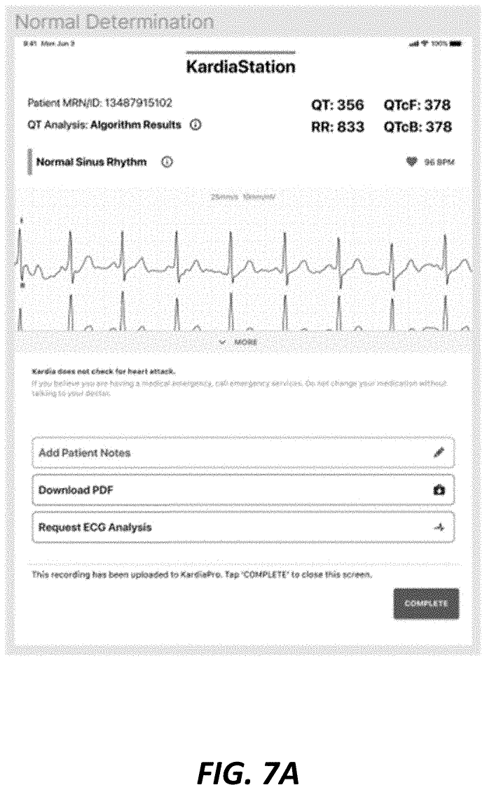

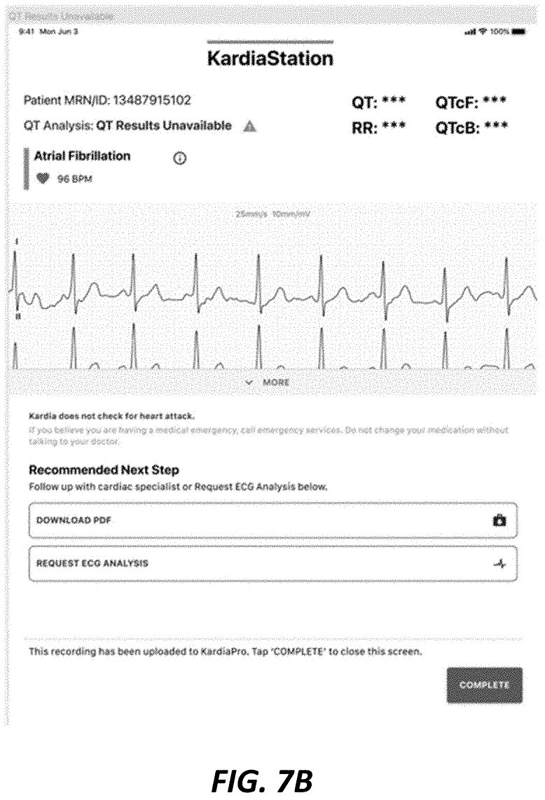

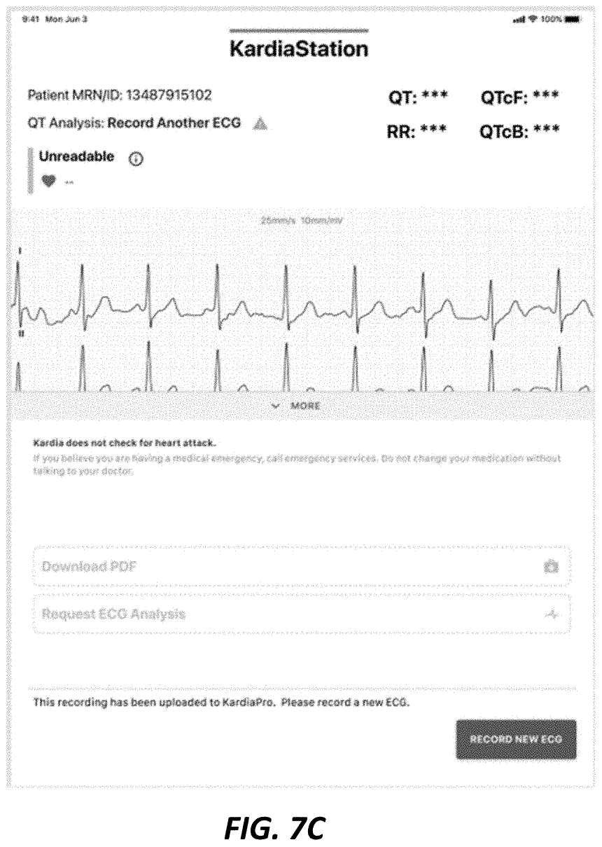

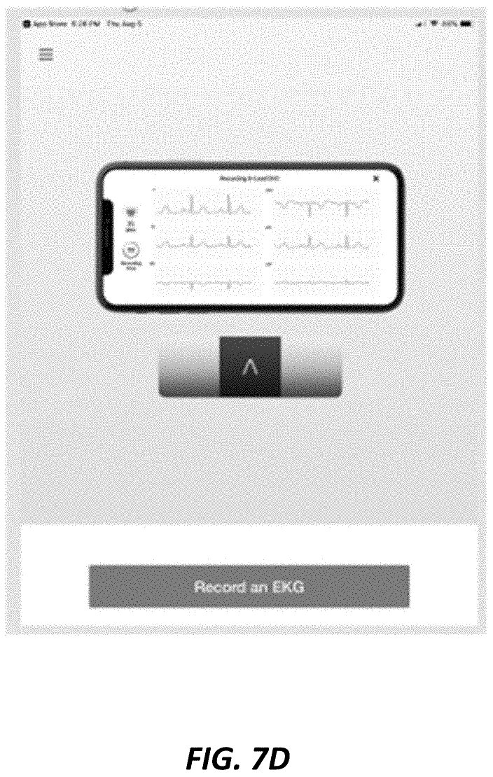

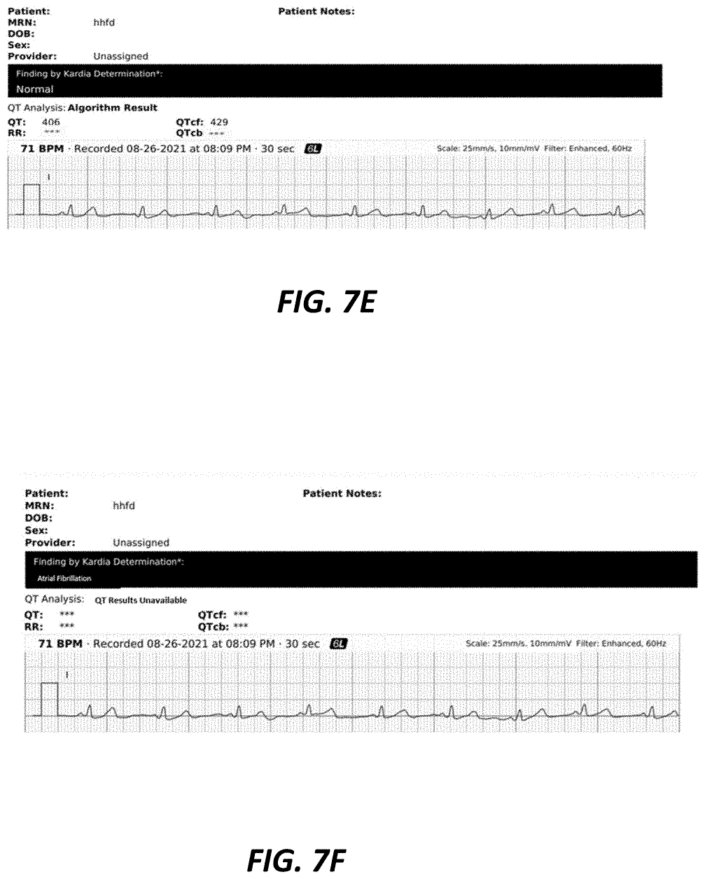

The ECG and QT analysis/management module 513A may account for various different results for both the QT analysis and the ECG measurements when synthesizing the GUI 600 for display. For example, when the QT analysis results is one of the following: normal sinus rhythm, tachycardia, bradycardia, sinus rhythm with premature ventricular contractions (PVCs), or sinus rhythm with supraventricular ectopy (SVE), the GUI 600 may be synthesized with additional information such as the RR and QTcB values of the patient, as depicted in FIG. 7A. In another example, when the QT analysis results is one of: unclassified, atrial fibrillation, or sinus rhythm with wide QRS, the GUI 600 may be synthesized as shown in FIG. 7B with the indication of the origin of the QT analysis results informing the healthcare professional that no QT results are available. FIG. 7C illustrates the GUI 600 when the ECG measurement is incomplete or when an error has occurred during measurement of the ECG. As can be seen, the indication of the origin of the QT analysis results informs the user that another ECG must be recorded.

As shown in FIGS. 7A and 7B, the GUI 600 may also include a “complete” button which may take the healthcare professional to a splash page such as shown in FIG. 7D where an option to begin a new recording (“record new EKG”) may be provided. In some embodiments, the results of the QT analysis may be requested as a PDF. FIG. 7E illustrates the QT analysis results when provided as a PDF, and FIG. 7F illustrates the results of the QT analysis when provided as a PDF when the results of the QT analysis are not available.

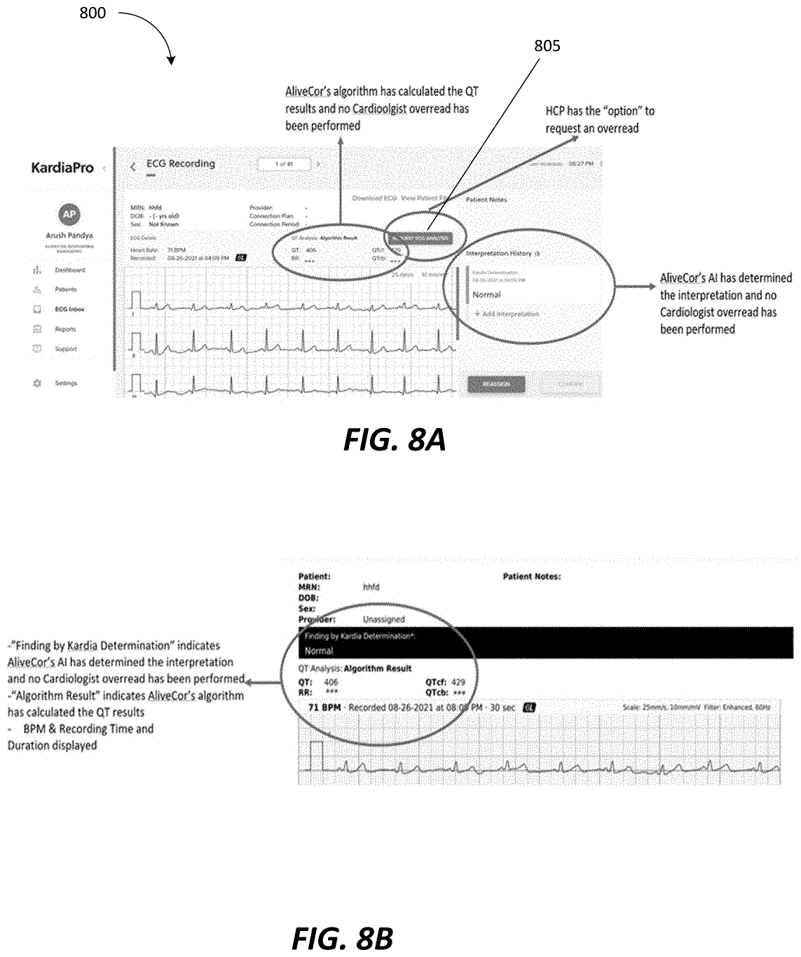

The analysis services module 524 may also provide a web portal via which the healthcare professional may log into the analysis services module 524 and input the patient's identifying information (e.g., MRN/ID) so as to access the patient record and the patient data 522 corresponding to the patient. Upon entering the patient's identifying information, the analysis services module 524 may provide a GUI 800 as shown in FIG. 8A which summarizes the patient record and displays the ECG data with integrated QT analysis.

At this point, the healthcare professional can download PDFs of the patient's ECG recording with integrated QT analysis and may manually upload it to a clinical database. FIG. 8B illustrates a sample PDF of the patient's ECG recording with integrated QT analysis but without a physician overread. Referring to FIG. 8B, the “Finding by Kardia Determination” indicates that the ECG and QT analysis/management module 513A has determined the interpretation and no cardiologist overread has been performed. The “Algorithm Result” status indicates that the QT analysis module 524 has calculated the QT results. The BPM and recording time and duration is also displayed.

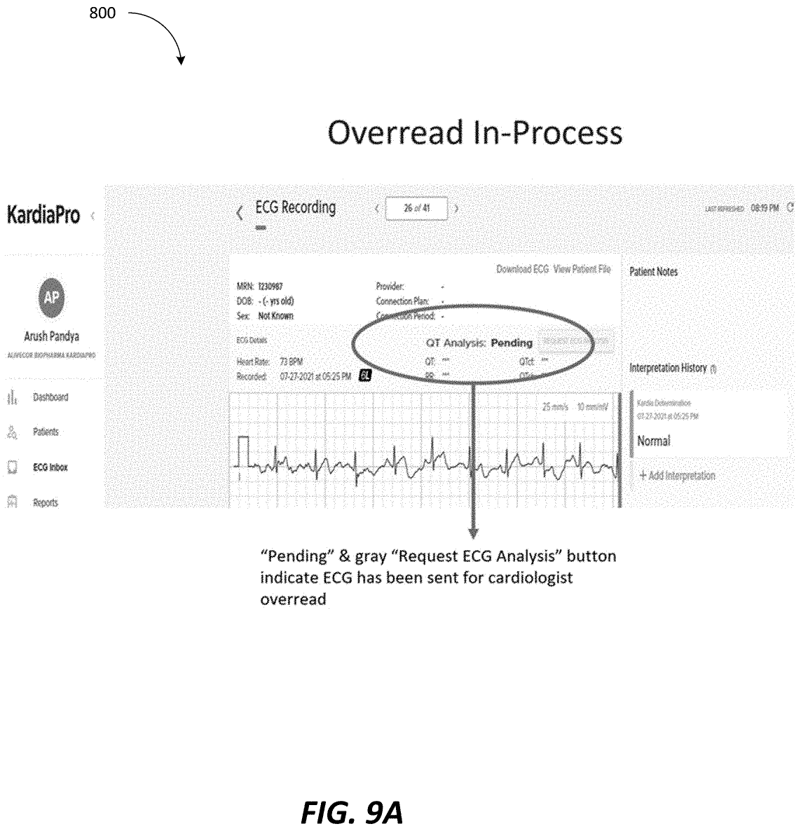

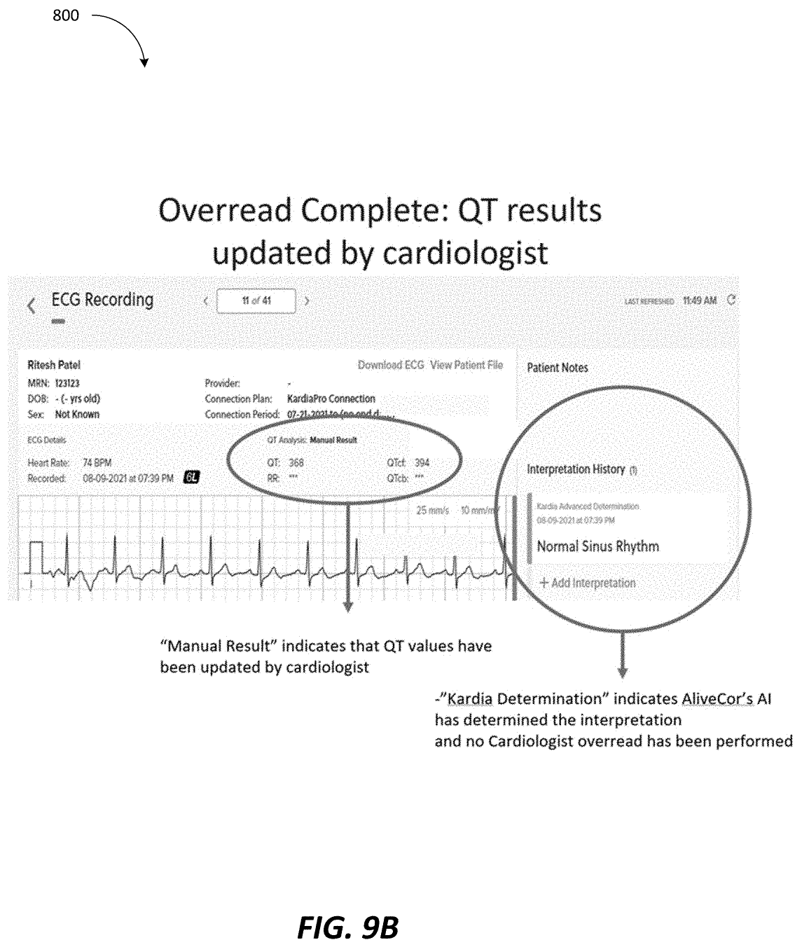

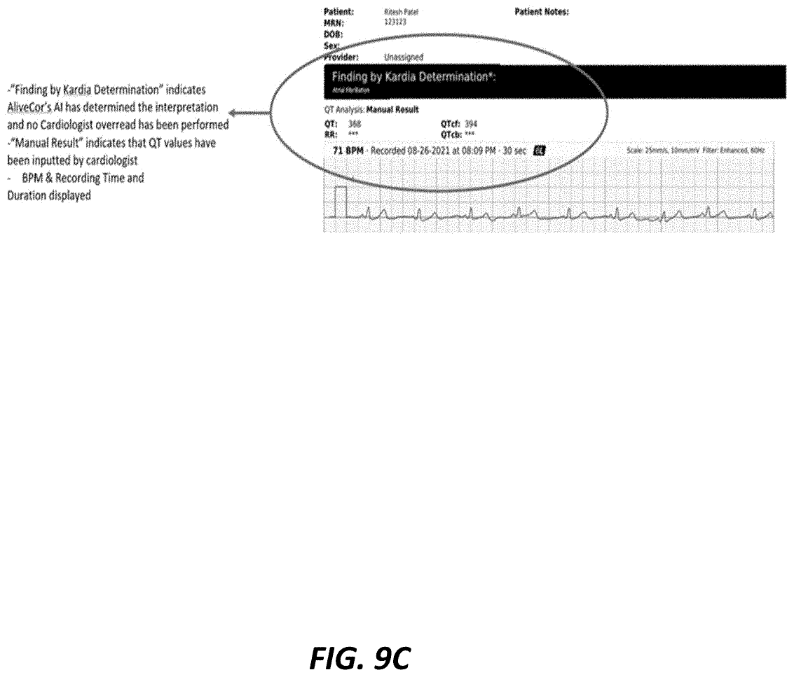

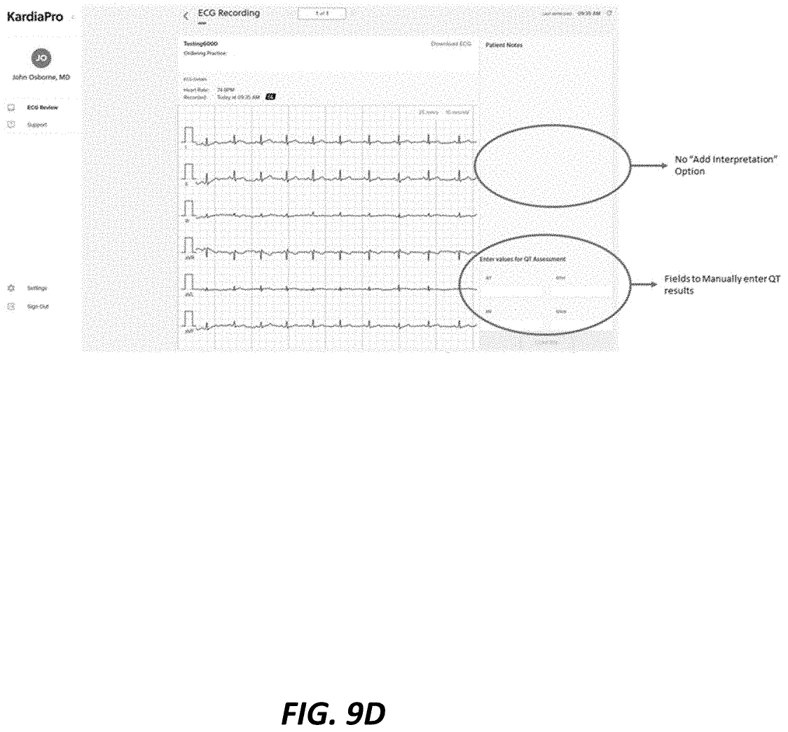

Referring back to FIG. 8A, the GUI 800 provides a button 805 labeled “request ECG analysis” via which the healthcare professional can request a physician overread. FIGS. 9A and 9B illustrate the GUI 800 during a request for a physician overread. As can be seen in FIG. 9A, the button 805 labeled “request ECG analysis” is now unavailable, indicating that the ECG data has been sent to a physician for an overread. FIG. 9B illustrates the GUI 800 once the overread has been completed. As can be seen in FIG. 9B, the indication of the origin of the QT analysis results now informs the health care provider that the QT analysis results are a manual result determined by a physician. FIG. 9C illustrates a PDF of the patient's ECG recording and integrated QT analysis results when a physician overread has been provided. FIG. 9D illustrates a GUI provided to a physician via which they may perform an overread.

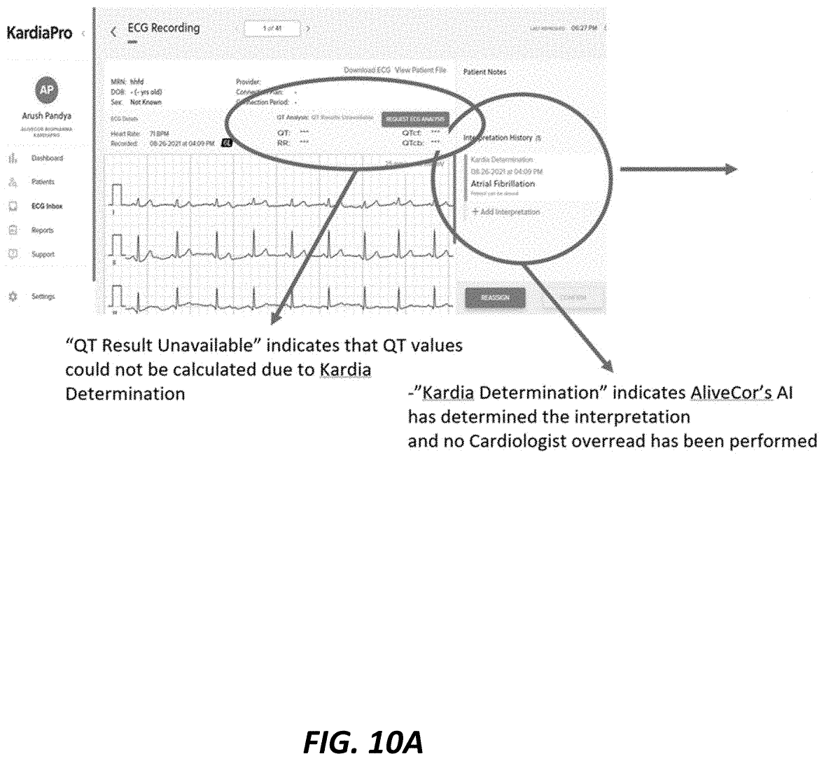

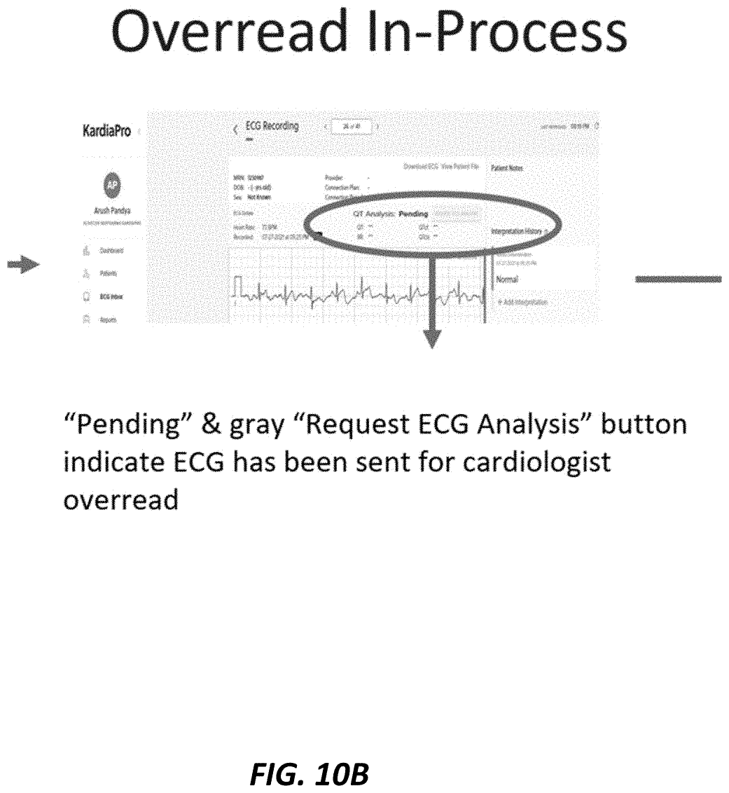

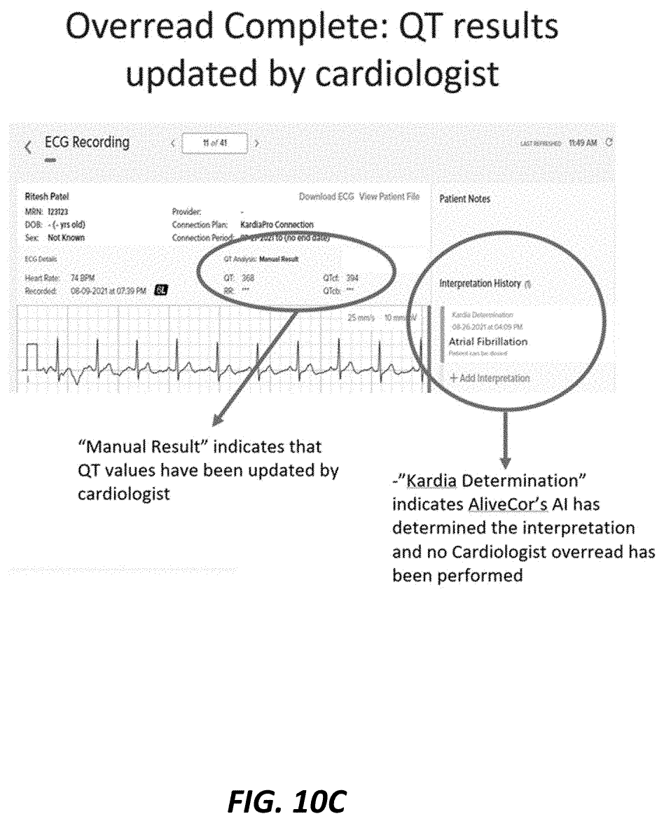

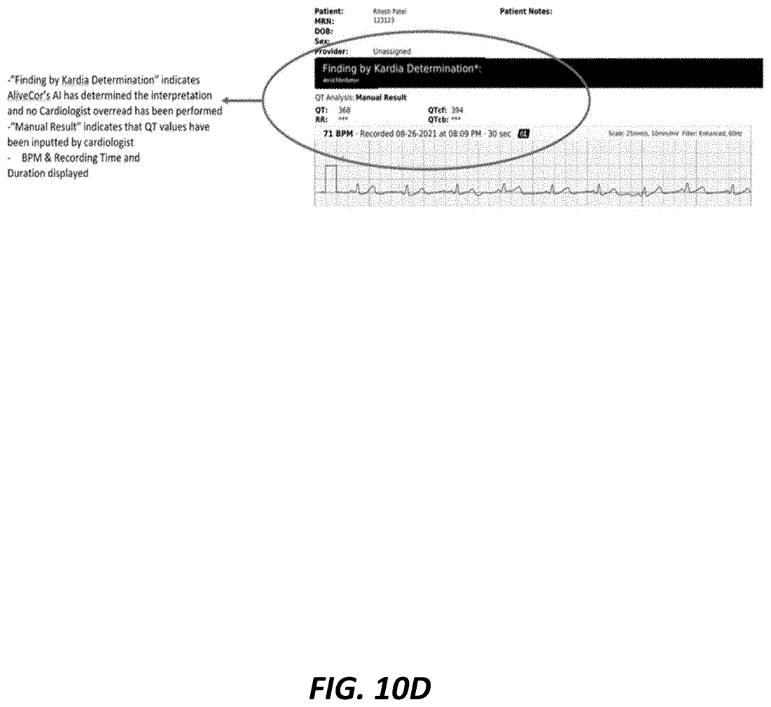

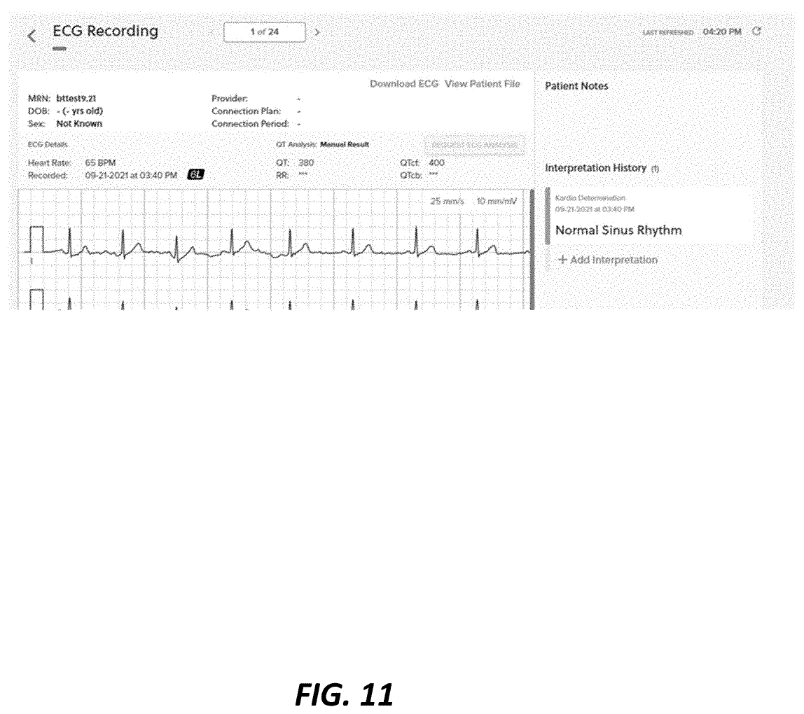

FIGS. 10A-10C illustrate the GUIs displayed by the analysis services module 524 when the QT results are unavailable and a manual result for the QT analysis has been uploaded. FIG. 10D illustrates a PDF of the results that can be downloaded by a healthcare professional when the QT results are unavailable and a manual result for the QT analysis has been uploaded. FIG. 11 illustrates a GUI displayed by the web portal of the analysis services module 524 when accessing a patient's data.

FIG. 12A shows top and bottom views of an exemplary ECG monitoring device 1200 comprising a set of electrodes 1202 (also referred to as an electrode assembly) in accordance with some embodiments of the present disclosure. In some embodiments, one or more capacitive electrodes are used in the ECG monitoring device 1200 so that, for example, the capacitive electrode senses an electric potential through a garment worn over the body of the user. Similarly, a conductive spray or gel may be placed on the body of the user so that a typical electrode senses an electric potential through a garment worn over the body of the user.

In one embodiment, the ECG monitoring device 1200 is constructed, in whole or in part, from stainless steel or some other suitable material. In one embodiment, the ECG device 1200 includes an exterior coating, such as Titanium Nitride or other suitable coating. Advantageously, such materials may increase biocompatibility and optimize electrode characteristics.

In one embodiment, the ECG monitoring device 1200 includes all necessary components to sense, record, and display ECG signals and analysis. In another embodiment, device 1200 connects via wires or wirelessly to a computing device (e.g., computing device 1212). In such a case, the device 1200 may sense the ECG signals and send the unmodified or modified signals to the computing device for further analysis and/or display. In yet another embodiment, any combination of the two examples listed above is possible. For example, although the ECG monitoring device 1200 may be considered a self-contained device, capable of performing all operations described herein, ECG monitoring device 1200 may still connect to, and interact with, a second computing device for any suitable purpose (offloading processing/analysis, display, etc.).

The ECG monitoring device 1200 may include one or more controls and/or indicators. For example, the device 1200 may include buttons, dials, etc. to select functions (e.g., turning on/off ECG reading, to begin to transmit ECG information, etc.). The ECG monitoring device 1200 may further include a display that displays a recorded ECG.

The ECG monitoring device 1200 may include a housing 1220, where two electrodes 1202A and 1202B are positioned on a top surface of the housing 520 and a third electrode 1202C is positioned on a bottom surface of the housing 1220 as shown in FIG. 12A. The electrodes 1202 may be insulated from each other via dialectrics 1204 or other suitable materials such that they are able to sense and record distinct signals. In some embodiments, the electrodes 1202 may be comprised of silver-silver chloride (or some other suitable material) electrodes. In some embodiments, ECG monitoring device 1200 may include an electrode connector (not shown) such as e.g., a female socket on one end or a side allowing one or more ECG electrodes to be connected to the ECG monitoring device 1200 to be used on skin with an adhesive or without an adhesive (e.g., a conductive gel and the electrodes 1202).

FIG. 12B illustrates a hardware block diagram of ECG monitoring device 1200, which may include hardware such as processing device 1205 (e.g., processors, central processing units (CPUs)), memory 1210 (e.g., random access memory (RAM), storage devices (e.g., hard-disk drive (HDD)), solid-state drives (SSD), etc.), and other hardware devices (e.g., analog to digital converter (ADC) etc.). A storage device may comprise a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices. In some embodiments, the processing device 1205 may comprise a dedicated ECG waveform processing and analysis chip that provides built-in leads off detection. The ECG monitoring device 1200 may include an ADC (not shown) having a high enough sampling frequency for accurately converting the ECG waveforms measured by the set of electrodes 1202 into digital signals (e.g., a 24 bit ADC operating at 500 Hz or higher) for processing by the processing device 1205.

The memory 1210 may include a lead synthesis software module 1210A (hereinafter referred to as module 1210A). The processing device 205 may execute the module 207A to synthesize ECG waveforms corresponding to leads that were not measured by the electrodes of the ECG monitoring device 1200 as discussed in further detail herein. The memory 1210 may include a time alignment software module (not shown) which can perform time alignment of the P, QRS, and T waveforms/complexes of each lead sensed by the ECG monitoring device 500 so that the sensed ECG leads are aligned when displayed as are the waveforms in a traditional ECG tracing. In some embodiments of the ECG monitoring device, the ECG monitoring device comprises a software application configured to time align two or more sensed ECG leads. In some embodiments of the ECG monitoring device, a software application configured to time align two or more sensed ECG leads is a component of a system that receives data from an ECG monitoring device.

The ECG monitoring device 1200 may further comprise a transceiver 1208, which may implement any appropriate protocol for transmitting ECG data wirelessly to one or more local and/or remote computing devices (e.g., computing device 512). For example, the transceiver 1208 may comprise a Bluetooth™ chip for transmitting ECG data via Bluetooth to local computing devices (e.g., a laptop or smart phone of the user). In other embodiments, the transceiver 1208 may include (or be coupled to) a network interface device configured to connect with a cellular data network (e.g., using GSM, GSM plus EDGE, CDMA, quadband, or other cellular protocols) or a WiFi (e.g., an 802.11 protocol) network, in order to transmit the ECG data to computing device 512 and/or another remote or local computing device.

As shown in FIG. 12C, in one practical example, a user holds the device with one or both hands so that each hand contacts an electrode 1202A and 1202B on the ECG monitoring device 1200 while the left leg contacts electrode 1202C. The ECG monitoring device 1200 (with, optionally, a separate computing device) may then be used to record Lead I, Lead II, and Lead III, from which at least three additional leads may be determined (e.g., by executing module 1210A), as described in further detail herein. Specifically, the augmented leads, aVR, aVL, and aVF, may be determined using Leads I, II, and III. The user may be sitting, standing, or in any position of comfort.

FIGS. 12D and 12E illustrate an embodiment where a user may also record the precordial leads V1, V2, V3, V4, V5, and V6 using the ECG monitoring device 1200 as described herein. A user may hold the ECG monitoring device 1200 so that each hand of the user contacts an electrode 1202A and 1202B while the third electrode (e.g., 1202C) is held against the chest so as to contact one of the six precordial chest positions which are represented as “CP1,” “CP2,” “CP3,” “CP4,” “CP5,” and “CP6”. For example, the user may start with the ECG monitoring device 1200 positioned such that electrode 1202C is contacting CP1 and from here, the user may move the ECG monitoring device 1200 such that it sequentially makes contact with each of the six electrode positions corresponding to leads V2, V3, V4, V5, and V6. In some embodiments, while the user contacts an electrode 1202A and 1202B of the ECG monitoring device 1200 with each of his right and left hands and simultaneously holds the third electrode (e.g., 1202C) of the device 1200 against a position on his chest corresponding to V1, V2, V3, V4, V5, and V6, each of the electric potentials sensed at the chest positions corresponding to V1, V2, V3, V4, V5, and V6 are sensed simultaneously with an electric potential sensed at LA and RA. Lead I is equivalent to the potential difference between LA and RA. Thus, in some embodiments, measuring an electric potential at a position on the chest corresponding to any of V1, V2, V3, V4, V5, and V6 together with the electric potential at the LA and RA positions is equivalent to the difference in potential at the chest position and lead I. That is, for example, using all three electrodes of device 1200 as described, V1 (the electric potential at the V1 chest position)=(“CP1”)−WCT (WCT=(RA+LA+LL)/3 or (lead I+lead II)/3).

The six precordial chest positions can be represented as (“CP1,” “CP2,” “CP3,” “CP4,” “CP5,” and “CP6”) and a composite value known as Wilson's Central Terminal (“WCT”). “CP(x)” corresponds to any of the six potentials sensed at the anatomical precordial lead positions (where “x” is a position number 1-6). For example, CP1 is the ECG measurement sensed at a location at which an electrode is placed to measure V1, and that position is approximately in the second intercostal space immediately to the right of the sternum. Thus, lead V1=CP−WCT.

WCT is equal to one third of the sum of the potentials sensed at the right upper extremity, left upper extremity, and left lower leg or 1/3(RA+LA+LL). In a standard ECG that uses ten simultaneously placed electrodes, a WCT value is generated at the same time that a precordial lead is sensed, because RA, LA, LL, which determine WCT, are sensed at the same time as CPI, CP2, CP3, CP4, CP5, and CP6.

In these embodiments, the electrodes 1202 are positioned and configured to simultaneously sense/calculate the six limb leads I, II, III, aVR, aVL, and aVF when a user contacts a first electrode 1202A with a right upper extremity, a second electrode 1202B with a left upper extremity, and a third electrode 1202C with a left lower extremity.

As also described herein, an ECG monitoring device 1200 is configured to sense the six leads V1, V2, V3, V4, V5, and V6 sequentially when a user, for example, contacts a first electrode 1202A with a right upper extremity, a second electrode 1202B with a left upper extremity, and a third electrode 1202C with an area of his or her chest corresponding to a precordial lead position.

In some embodiments in which the ECG monitoring device 1200 comprises three electrodes as described herein, the RA, LA, LL, which determine WCT, are not sensed simultaneously with one or more precordial leads. That is, when one of the three electrodes of the ECG monitoring device 1200 is held against the chest wall of a user, only two electrodes remain free and a traditional WCT cannot be simultaneously determined. In some of these embodiments, RA is set to 0. When RA=0, it provides a WCT=(0+LA+LL)/3 or ((LA−0)+(LL−0))/3 which can be further expressed as WCT=(lead I+lead II)/3.

Likewise, in these embodiments, wherein RA is set to 0, an averaged WCT=(averaged lead I+averaged lead II)/3. An averaged WCT in some embodiments is generated using an averaged lead I and an averaged lead II that are generated using, for example, an ensemble averaging method on the lead I and lead II waveforms sensed by the ECG monitoring device described herein. Generating an average WCT is beneficial in, for example, signal filtering and also simplifies alignment of values for purposes of subtraction. That is, in some embodiments, CPI, CP2, CP3, CP4, CP5, and CP6 are each averaged and an averaged WCT is respectively subtracted from each to generate V1, V2, V3, V4, V5, and V6.

A number of machine learning (ML) methods may also be used to synthesize the full 12 lead set from the set of leads measured by the ECG monitoring device 500. ML is well suited for continuous monitoring of one or multiple criteria to identify anomalies or trends, big and small, in input data as compared to training examples used to train the model. The ML model described herein may be trained on user data from a population of users, and/or trained on other training examples to suit the design needs for the model. ML models that may be used with embodiments described herein include by way of example and not limitation: Bayes, Markov, Gaussian processes, clustering algorithms, generative models, kernel and neural network algorithms. Some embodiments utilize a machine learning model based on a trained neural network (e.g., a trained recurrent neural network (RNN) or a trained convolution neural network (CNN)).

For example, the ML model may utilize artificial neural networks (ANNs) for supervised classification, where the outcome of the model represents the probability of the input sample to be in a specific class of data or exhibits some peculiar characteristics. In another example, a data driven approach based on convolutional neural networks (CNNs) is used. By using convolution operations, the ML model may take into account the correlation among temporally closed input samples to infer a single output data point. More specifically, a single output sample (each precordial lead) at a generic time t is affected by all the input samples (all limb leads) from t−τ to t+τ. The value of τ, which represents the receptive field of the network, highly depends on the model architecture and typically increases with its depth, i.e., the number of consecutive layers. The ability to generalize on unseen data, and avoid overfitting issues, is of primary importance for all data driven approaches. Complex models, along with small datasets, may lead to excellent performance on the training set, but may perform poorly on unseen data. Any appropriate regularization method may be used to optimize the model, such as inter and intra-layer normalization (e.g., batch normalization and layer normalization), and data augmentation techniques. Finally, to improve the effectiveness and efficiency of the model, the use of residual connections, i.e., an identity mapping that allow gradients to flow through a layer during the backpropagation of gradient-based optimization algorithms may be utilized.

The use of AI/deep learning with multi-lead ECG monitoring devices may allow patients themselves (in hospital or at home) to monitor the electrical activity of their heart without the need for hospital visits or bulky hardware.

In some embodiments, the memory 1210 of the ECG monitoring device 1200 or another computing device (e.g., computing device 512) may include an instruction software module (not shown) that displays or otherwise transmits instructions to an individual instructing the user as to how to position the ECG monitoring device 1200 in order to perform an ECG (e.g., over the standard precordial lead chest positions) as well as a position in which the user should be situated in order to perform an ECG. For example, a display may show an image of a location on the user's chest against which the user is instructed to hold the third electrode while holding electrodes one and two with his left and right hands respectively.

In some embodiments, software on the ECG monitoring device 1200 or computing device 512 is configured to recognize if a first electrode is contacted by a left hand and second electrode is being contacted by a right hand versus whether a first electrode is contacted by a right hand a second electrode is contacted by a left hand. For example, in some embodiments, a third electrode is positioned on a different surface of the ECG monitoring device 1200 than the first and second electrodes, such that a user will likely need to swap hand positions to contact the precordial lead positions on their chest with the third electrode after contacting their left leg with the third electrode. In some embodiments, software on the ECG monitoring device 1200 or other computing device receives information from a sensor coupled with or integrated with an ECG monitoring device 1200, wherein the sensor provides information about the position of the device in space. Examples of the class of sensors that sense such information include but are not limited to accelerometers, inclinometers, and gyrometers.

In some embodiments, the ECG monitoring device 1200 is configured to sense an ECG when one or more of the electrodes 1202 are not engaged by the user. For example, in some embodiments, the ECG monitoring device 1200 comprises three electrodes, and the ECG monitoring device 1200 is configured to sense an ECG when either all three electrodes are engaged by the user or when any two of the three electrodes are engaged by the user. That is, in this embodiment, when a user, for example, contacts a skin surface on their right upper extremity with a first electrode and contacts a skin surface on their left upper extremity with a second electrode, but does not contact the third electrode, the ECG monitoring device senses an ECG. When, in this example, the two of three electrodes are contacted by a right and left upper extremity respectively, a lead I is sensed. Likewise, when the two of three electrodes are contacted by a right upper extremity and left lower extremity respectively, a lead II is sensed. Likewise, when the two of three electrodes are contacted by a left upper extremity and left lower extremity respectively, a lead III is sensed. In this embodiment, the ECG monitoring device 1200 recognizes that one or more of the electrodes have not been contacted by a user while two or more electrodes have been contacted by the user, by, for example, sensing an electrode potential from two or more electrodes that are contacted but not sensing an electrode potential from electrodes that are not contacted by the user.

In some embodiments of the ECG monitoring device 1200 described herein, exemplary embodiments of which are shown in FIGS. 12A-12E, the ECG monitoring device 1200 is configured to run a software application as described herein. In further embodiments, the ECG monitoring device 1200 includes one or more hardware central processing units (CPUs) or general purpose graphics processing units (GPGPUs) that carry out the device's functions. In some embodiments, the ECG monitoring device 1200 is optionally connected a computer network. In further embodiments, the ECG monitoring device 1200 is optionally connected to the Internet such that it accesses the World Wide Web. In still further embodiments, the ECG monitoring device 1200 is optionally connected to a cloud computing infrastructure. In other embodiments, the ECG monitoring device 1200 is optionally connected to an intranet. In other embodiments, the ECG monitoring device 1200 is optionally connected to a data storage device.

In accordance with the description herein, suitable computing devices include, by way of non-limiting examples, server computers, desktop computers, laptop computers, notebook computers, sub-notebook computers, netbook computers, netpad computers, handheld computers, smartphone, smartwatches, digital wearable devices, and tablet computers.

In some embodiments, the ECG monitoring device 1200 includes a storage and/or memory device. The storage and/or memory device is one or more physical apparatuses used to store data or programs on a temporary or permanent basis. In some embodiments, the storage and/or memory device is volatile memory and requires power to maintain stored information. In some embodiments, the storage and/or memory device is non-volatile memory and retains stored information when the ECG monitoring device 1200 is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes, optical disk drives, and cloud computing based storage. In further embodiments, the storage and/or memory device is a combination of devices such as those disclosed herein.

All of the devices described herein are suitable for use in various systems, which may include one or more servers, one or more sensors, an electronic data communication networks, as well as other ECG monitoring devices. In some embodiments, a plurality of ECG monitoring devices as described herein transmit ECG data to one or more remote servers through an electronic data communication network. In some embodiments, the ECG data is analyzed using the one or more remote servers. In some embodiments, arrhythmia detection is carried out using a remote server that analyzes received ECG data.

All of the devices and systems described herein may also include one or more software modules. In some embodiments, software comprises an application that is configured to run on a computing device such as, for example, a smartphone, a smartwatch, or a tablet computer. The software receives and processes ECG data received from an ECG monitoring device. The software identifies separate leads within the transmitted data, based on for example, which electrodes the ECG data originated from. For example, the software may be able to identify a lead I based on the signal originating from two electrodes that measure an electric potential difference between the right and left upper extremities. Once an ECG is identified, the software may further be configured to display a single or multi-lead ECG on a display screen of a computing device. The software may be configured to display six leads I, II, III, aVR, aVL, and aVF simultaneously on a display screen. The software may be configured to display one or more of the six leads I, II, III, aVR, aVL, and aVF on a display screen at once, wherein a user is able to manually toggle screens to see a different lead or leads on different toggled screens.

In some embodiments, two or more sensed leads that are not simultaneously sensed are time aligned to generate a time aligned ECG tracing displaying two or more leads in a time aligned format such as in a traditional standard twelve lead ECG tracing. In some embodiments of the ECG monitoring device described herein, one or more ECG sensing electrodes are not simultaneously positioned on the skin of the individual whose ECG is sensed (i.e. some leads may be sequentially sensed). For example, the limb leads (I, II, III, aVR, aVL, and aVF) are simultaneously sensed while one or more of the precordial leads are sensed separately from the limb leads. As such, in these embodiments, the six limb leads are not automatically time aligned with the individually and separately sensed precordial leads and a further process is carried out by a software application to time align one or more of the limb leads with one or more of the precordial leads. In some embodiments, one or more of the six precordial leads are individually sensed so that the individually sensed precordial leads are time aligned by a software application with the six limb leads as well as with the other precordial leads. In some embodiments, a software application described herein aligns two or more sensed precordial leads with one another and separately time aligns six sensed limb leads so that two sets of six leads are respectively time aligned (i.e. six time aligned precordial leads and six separately time aligned limb leads). In some embodiments, the software described herein aligns two or more sensed precordial leads with one another as well as with sensed limb leads so that all twelve sensed leads are time aligned.

In some embodiments, one or more average or median waveforms are generated for a first and a second lead so that waveforms corresponding to different heartbeats are time aligned. That is, in some embodiments wherein one or more leads are not sensed concurrently, an average or median waveform is generated for one or more of these leads and the averaged or median waveforms are time-aligned so that the P, QRS, and T waveforms/complexes are aligned vertically along the X-axis.

When first and second electrodes of the ECG monitoring device described herein are contacted by the right and left upper extremities of the user at the same time that a third electrode of the device contacts any one of the six precordial lead positions, a lead I is sensed simultaneously along with a sensed precordial lead. That is, lead I is equal to a voltage sensed at the left upper extremity minus a voltage sensed at the right upper extremity, so when left upper extremity, right upper extremity, and chest are all respectively contacted by an electrode of the ECG monitoring device described herein, a lead I is sensed in addition to a precordial lead. Therefore, when all six precordial leads are sensed sequentially, six respectively corresponding “precordial lead I recordings” are also generated: V1-lead I, V2-lead I, V3-lead I, V4-lead I, V5-lead I, and V6-lead I. Each of these six precordial lead I recordings is used to time align each of the precordial leads to the limb leads and thus time aligns precordial leads.

In some embodiments, the time alignment software module aligns the precordial leads V1, V2, V3, V4, V5, and V6 by taking advantage of there being precordial lead I recordings sensed simultaneously with each of the V1, V2, V3, V4, V5, and V6 waveforms. That is, the precordial lead I recordings V1-lead I, V2-lead I, V3-lead I, V4-lead I, V5-lead I, and V6-lead I are each respectively time aligned with a precordial lead recording with which they are simultaneously sensed. Each of the precordial lead I recordings is time aligned with the lead I that is sensed along with the limb leads, by, for example, moving the precordial lead I recording a certain distance along the Y-axis, and because each of the precordial lead I recordings is time aligned with a precordial lead, each of the respective precordial leads V1, V2, V3, V4, V5, and V6 will also be time aligned when moved the same distance along the Y-axis as their co-sensed precordial lead I recording. For example, “V1-lead I” is a lead I recording that is time aligned with V1. “V1-lead I” is not the same as “lead I,” which is the lead I recorded simultaneously sensed with the other five limb leads using the ECG monitoring device described herein. “V1-lead I” is also not necessarily time aligned with “lead I” as these two different lead I recordings are not typically sensed simultaneously using the ECG monitoring device described herein. Because, however, “V1-lead I” and “lead I” are both lead I recordings, they can be time aligned in a fairly straightforward manner as they would both be expected, when averaged, to have very similar (if not identical) morphology and timing between waveforms. For example, if the peak of the R wave of an averaged “lead I” occurs at 1 second, and the peak of the R wave of an averaged “V1-lead I” occurs at 1.5 seconds, the averaged “V1-lead I” will be re-positioned or shifted 0.5 seconds along the Y-axis so that the peak of its R wave occurs at 1 second as it does in in the averaged “lead I.” Because V1 is time aligned with V1-lead I, it too must be shifted 0.5 seconds along the Y-axis in order to time align it with the averaged “lead I.” When V1 is time aligned with “lead I,” it will also be time aligned with the other five limb leads that are already time aligned with “lead I.” A similar alignment occurs with V2, V3, V4, V5, and V6 by respectively aligning V2-lead I, V3-lead I, V4-lead I, V5-lead I, and V6-lead I with “lead I.”

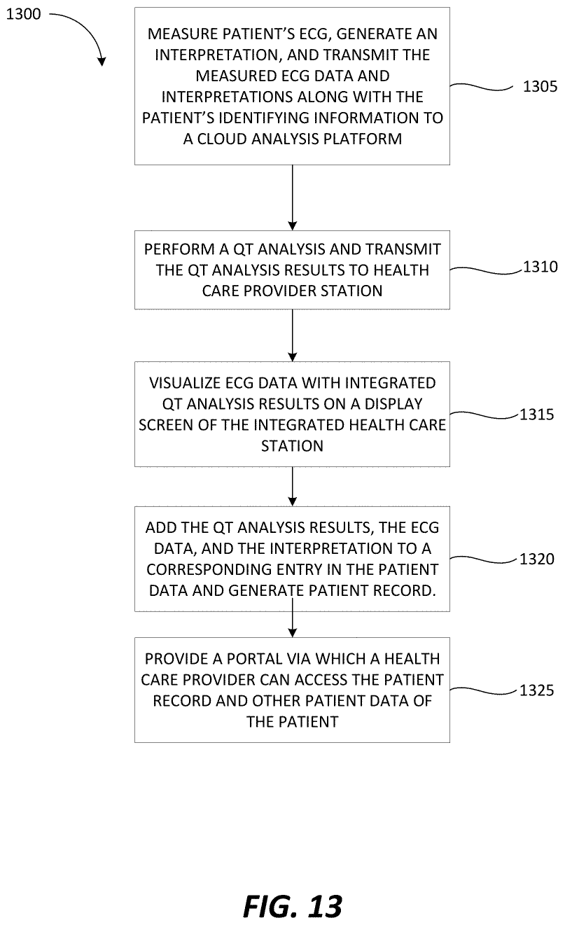

FIG. 13 is a flow diagram of a method 1300 for visualizing ECG data with QT interval analysis results integrated therein, in accordance with some embodiments of the present disclosure. Method 1300 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the method 1300 may be performed by computing device 512, ECG monitoring device 514, and cloud computing/storage service 520, as illustrated in FIG. 5.

At block 1305, the ECG monitoring device 514 may perform an ECG of the patient and determine ECG data of the patient and generate an interpretation thereof. The ECG monitoring device 514 may transmit the ECG data and interpretation (along with the patient's identifying information) to the cloud analysis platform 520. In some embodiments, the ECG monitoring device 514 may transmit the ECG data and interpretation (along with the patient's identifying information) to the computing device 512 for transmission to the cloud analysis platform 520.

Upon receiving the ECG data and interpretation (along with the patient's identifying information), at block 1310 the cloud analysis platform 520 may execute the QT analysis module 526 in order to perform a QT analysis and generate QT analysis results. The QT analysis results may include the patient's QT and QTcF values. The cloud analysis platform 520 may transmit the QT analysis results to the computing device 512. At block 1315, the computing device 512 may visualize the ECG data and QT analysis results using an improved graphical user interface (GUI) for visualizing the ECG data with the QT analysis results integrated therein. The memory 512B of the computing device 512 may include an ECG and QT analysis/management module 513A which may include logic for providing the improved graphical user interface (GUI) for visualizing the ECG data with the QT analysis results integrated. The GUI for visualizing the ECG data with integrated QT analysis results may be displayed on a display of the computing device 512.

At block 1320, the analysis services module 524 may use the patient identifier (e.g., MRN/ID provided along with the ECG data by the ECG monitoring device 514) to add the QT analysis results, the ECG data, and the interpretation to a corresponding entry in the patient data 522. The analysis services module 524 may also append a limited set of patient information (e.g., first and last name, email address, and date of birth in the example of FIG. 5) to the QT analysis results, the ECG data, and the interpretation to generate a patient record. At block 1325, the cloud analysis platform 520 may provide a portal via which a health care provider can access the patient record and other patient data 522 of the patient via the computing device 512 of the integrated health care station 510 as discussed in further detail herein. The healthcare professional can download PDFs of the patient record and may manually upload it to a clinical database.

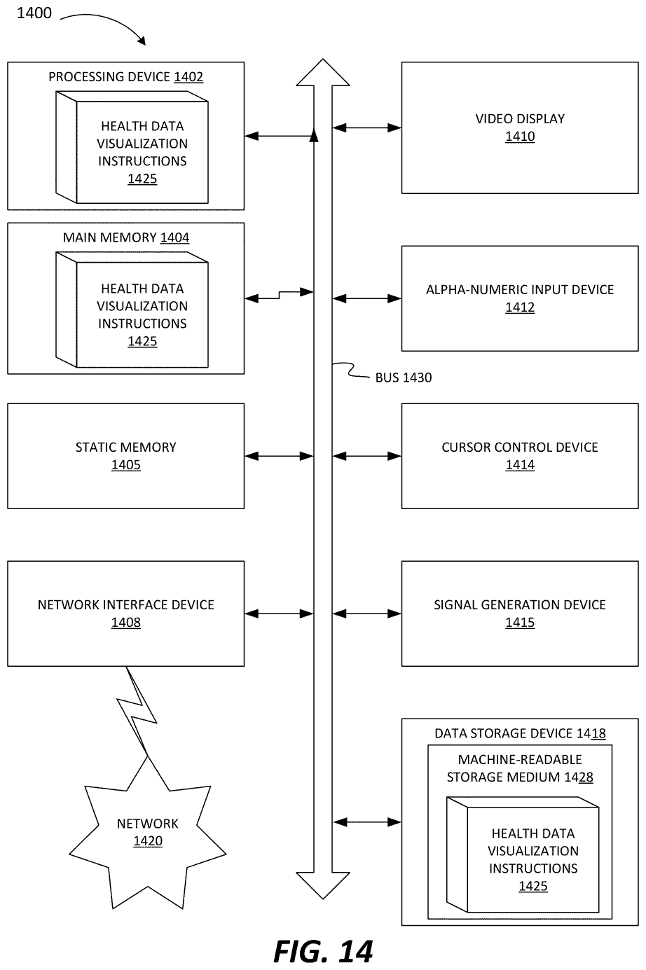

FIG. 14 is a block diagram of an example computing device 1400 that may perform one or more of the operations described herein, in accordance with some embodiments. In some embodiments, computing device 1400 may be similar to computing device 512. Computing device 1400 may be connected to other computing devices in a LAN, an intranet, an extranet, and/or the Internet. The computing device may operate in the capacity of a server machine in client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein.

The example computing device 1400 may include a processing device (e.g., a general purpose processor, a PLD, etc.) 1402, a main memory 1404 (e.g., synchronous dynamic random access memory (DRAM), read-only memory (ROM)), a static memory 1406 (e.g., flash memory and a data storage device 1418), which may communicate with each other via a bus 730.

Processing device 1402 may be provided by one or more general-purpose processing devices such as a microprocessor, central processing unit, or the like. In an illustrative example, processing device 1402 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 1402 may also comprise one or more special-purpose processing devices such as an application specific integrated circuit (ASIC), a field programmable gate array (FPGA), a digital signal processor (DSP), network processor, or the like. The processing device 1402 may be configured to execute the operations described herein, in accordance with one or more aspects of the present disclosure, for performing the operations and steps discussed herein.

Computing device 1400 may further include a network interface device 1408 which may communicate with a network 1420. The computing device 1400 also may include a video display unit 1410 (e.g., a liquid crystal display (LCD) or a cathode ray tube (CRT)), an alphanumeric input device 1412 (e.g., a keyboard), a cursor control device 1414 (e.g., a mouse) and an acoustic signal generation device 1416 (e.g., a speaker). In one embodiment, video display unit 1410, alphanumeric input device 1412, and cursor control device 1414 may be combined into a single component or device (e.g., an LCD touch screen).

Data storage device 1418 may include a computer-readable storage medium 1428 on which may be stored one or more sets of health data visualization instructions 1426, e.g., instructions for carrying out the operations described herein, in accordance with one or more aspects of the present disclosure. Health data visualization instructions 1426 may also reside, completely or at least partially, within main memory 1404 and/or within processing device 1402 during execution thereof by computing device 1400, main memory 1404 and processing device 1402 also constituting computer-readable media. The health data visualization instructions 1426 may further be transmitted or received over a network 1420 via network interface device 1408.

While computer-readable storage medium 1428 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

The methods and illustrative examples described herein are not inherently related to any particular computer or other apparatus. Various general purpose systems may be used in accordance with the teachings described herein, or it may prove convenient to construct more specialized apparatus to perform the required method steps. The required structure for a variety of these systems will appear as set forth in the description above.

The above description is intended to be illustrative, and not restrictive. Although the present disclosure has been described with references to specific illustrative examples, it will be recognized that the present disclosure is not limited to the examples described. The scope of the disclosure should be determined with reference to the following claims, along with the full scope of equivalents to which the claims are entitled.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on). The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. 112, sixth paragraph, for that unit/circuit/component. Additionally, “configured to” or “configurable to” can include generic structure (e.g., generic circuitry) that is manipulated by software and/or firmware (e.g., an FPGA or a general-purpose processor executing software) to operate in manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed device, unless accompanied by programmed media that confers the ability to the unprogrammed device to be configured to perform the disclosed function(s).

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalents of the appended claims.

What is claimed is:

1. A system comprising:
an electrocardiogram (ECG) monitoring device to:
measure an ECG of a user to generate ECG data; and
transmit the ECG data;
a cloud analysis platform to:
receive the ECG data;
perform a QT analysis based on the ECG data to generate QT analysis results; and
transmit the QT analysis results; and
a computing device to:
receive the QT analysis results; and
provide a graphical user interface (GUI) to visualize the ECG data with the QT analysis results integrated therein.

2. The system of claim 1, wherein the GUI comprises:
an indication of an origin of the QT analysis results;
an indication of the QT analysis results; and
a visualization of the ECG data.

3. The system of claim 2, wherein the QT analysis results comprise:
a QT value; and
a QTcF value.

4. The system of claim 2, wherein the GUI further comprises:
a feature to request a portable document format version of the visualization of the ECG data and the QT analysis results.

5. The system of claim 2, wherein the ECG monitoring device is further to:
generate an interpretation of the ECG data; and
transmit the interpretation along with the ECG data to the cloud analysis platform.

6. The system of claim 5, wherein the cloud analysis platform is further to:
add the QT analysis results, patient data, the interpretation, and the ECG data to a patient record.

7. The system of claim 5, wherein the GUI further comprises an indication of the interpretation.

8. The system of claim 5, wherein the patient data comprises date of birth, height, weight, sex, other health conditions, and previous ECG data.

9. The system of claim 6, wherein the cloud analysis platform is further to:
provide a portal through which patient records can be accessed.

10. The system of claim 5, wherein the ECG monitoring device further transmits a patient identifier along with the ECG data to the cloud analysis platform.

11. A method comprising:
measuring an electrocardiogram (ECG) of a user to generate ECG data;
transmitting the ECG data to a cloud analysis platform configured to perform a QT analysis based on the ECG data to generate QT analysis results;
receive the QT analysis results; and
provide a graphical user interface (GUI) to visualize the ECG data with the QT analysis results integrated therein

12. The method of claim 11, wherein the GUI comprises:
an indication of an origin of the QT analysis results;
an indication of the QT analysis results; and
a visualization of the ECG data.

13. The method of claim 12, wherein the QT analysis results comprise:
a QT value; and
a QTcF value.

14. The method of claim 12, wherein the GUI further comprises:
a feature to request a portable document format version of the visualization of the ECG data and the QT analysis results.

15. The method of claim 12, further comprising:
generating an interpretation of the ECG data; and
transmitting the interpretation along with the ECG data to the cloud analysis platform.

16. The method of claim 15, wherein the cloud analysis platform is further configured to:
add the QT analysis results, patient data, the interpretation, and the ECG data to a patient record.

17. The method of claim 15, wherein the GUI further comprises an indication of the interpretation.

18. The method of claim 15, wherein the patient data comprises date of birth, height, weight, sex, other health conditions, and previous ECG data.

19. The method of claim 16, further comprising:
accessing patient records through a portal provided by the cloud analysis platform.

20. The method of claim 15, further comprising:
transmitting a patient identifier along with the ECG data to the cloud analysis platform.
